(19) United States
(12) Patent Application Publication
(10) Pub. No.: US 2026/0179747 A1
(43) Pub. Date: Jun. 25, 2026

(54) SYSTEM AND METHOD FOR CREATING AND MANAGING TREATMENT PLAN

(71) Applicants: Roderick L. Brown, Southfield, MI (US); Dwayne E. Goldsmith, Northville, MI (US); Vassal N. Johnson, Southfield, MI (US)

(72) Inventors: Roderick L. Brown, Southfield, MI (US); Dwayne E. Goldsmith, Northville, MI (US); Vassal N. Johnson, Southfield, MI (US)

(73) Assignee: ASENDIO, Inc, Ann Arbor, MI (US)

(21) Appl. No.: 19/434,265

(22) Filed: Dec. 29, 2025

Related U.S. Application Data
(60) Provisional application No. 63/738,672, filed on Dec. 24, 2024.

(51) Int. Cl. G16H 20/10; G16H 80/00

(52) U.S. Cl. CPC G16H 20/10; G16H 80/00

(57) ABSTRACT

A system and method for creating and managing treatment plan is disclosed. The system comprises one or more user devices associated with client, clinician, and administrator, a database, and a computing device in communication with database and user device via a network. The system is configured to enable the client to assess a treatment plan and to upload the assessed treatment plan comprising input data. The system is further configured to enable the counselor to review the assessed treatment plan and conduct an evaluation session with the client, generate first score data based on the input data and a second score data based on the evaluation session and the first score data, integrate the treatment plan into a master treatment plan for the client and enable the counselor to digitally sign the master treatment plan and enable the client to approve the master treatment plan using an electronic signature.

### CROSS REFERENCE TO RELATED APPLICATION

This application claims the priority benefit of the U.S. Patent Application No. 63/738,672, titled “SYSTEM AND METHOD FOR MANAGING TREATMENT PLANS FOR SUBSTANCE USE DISORDER”, filed on Dec. 24, 2024, the contents of which are hereby incorporated by reference in their entirety.

### TECHNICAL FIELD

The present invention generally relates to the monitoring and tracking of substance use disorder (SUD). More particularly, the invention is related to a system and method for managing treatment plans for substance use disorder (SUD).

### BACKGROUND

Substance Use Disorder (SUD) is a complex medical condition in which an individual has a diminished ability to control the use of one or more legal or illegal substances. Further, the substances could be alcohol, drugs, or prescription medications. The SUD further involves a range of behavioral, cognitive, and physiological issues, including cravings, inability to control substance use, and withdrawal symptoms. Further, repeated usage of substances could affect the brain function system.

SUD could vary in severity from mild to severe with mental health disorders, for example, anxiety or depression. The disorder could impact people and lead to significant personal, social, and economic consequences. Further, the SUD needs to be monitored to provide the required support to manage the potential risk of SUD individuals. Further, a few patents reference related to the system managing the treatment for SUD individuals are discussed as follows.

US20230290516 of Mark Matthews et. al., entitled “Adaptive and configurable delivery of measurement-based care to assess behavioral health status” discloses a computer-implemented method for optimal measurement-based care (MBC) assessment of the mental health conditions. The computer-implemented method executed using one or more computing devices. The computer implemented method involves receiving a measurement-based care (MBC) assessment request from a patient via a mobile computing device. The method then asynchronously assesses a plurality of MBC assessment data from the patient using an application server computer. The MBC assessment data includes passively collected values, assessments, programmed heuristics, programmed rules, and model thresholds. Further, the computer implemented method utilizes the application server computer and a machine learning model to asynchronously generate a set of vital sign values for the patient based on the MBC assessment data. Additionally, the system asynchronously determines stratification group data for the patient using the MBC assessment data via the machine learning model and the application server computer. Further, one or more instructions are sent to the mobile computing device via a user interface displaying stratification group data for the patient.

US20230343421 of Daniel Larson et. al., entitled “Centralized platform for management of substance abuse treatment programs” discloses the platform for management of substance abuse treatment programs implemented on a computing system comprises a centralized datastore. The centralized datastore is configured to manage a plurality of recovery users. Further, the datastore is configured to store demographic and personal information, preferences, treatment status, peer recovery certifications, related recovery users, and transaction history for each recovery user. The platform further comprises a plurality of data interfaces that is accessible by and from third party systems. The third-party system includes medical and counseling centers, government officers, and third-party entities authorized by a recovery user. Further, the plurality of data interfaces is configured to receive treatment and employment records from the third-party systems. The platform further comprises a plurality of user interfaces that are presented to the recovery user. The user interfaces include one or more user certification interfaces that display the treatment status of the recovery user.

However, the existing system lacks the ability to analyze factors affecting the substance use disorder (SUD) individual, including environmental, psychological, treatment consistency, and social influences, which could limit the comprehensiveness of patient data evaluations. The system further fails to consistently monitor the health of individuals with SUD across different counselors and organizations. The lack of consistency in monitoring could negatively affect the adjustment of treatment plans for SUD individuals. Additionally, the system relying on third-party systems for data input introduces risks of inaccuracies, inconsistencies, or delays in managing the treatment plan.

Therefore, there is a need for a system and method for managing treatment plans for individuals related to substance use disorder (SUD). The system needs to analyze the important factors affecting individuals with substance disorder use (SUD) to provide comprehensiveness patient data evaluations. Additionally, the system needs to be capable of monitoring the health status of individuals with SUD across multiple counselors to ensure consistency in assessment and enabling dynamic adjustments to individualized treatment plans.

### SUMMARY

The present invention discloses a system and method for creating and managing a treatment plan. The system comprises one or more user devices, including at least one first user device associated with a client, at least one second user device associated with a clinician and a counselor, and at least one third user device associated with an administrator. The clinician is also referred to as the counselor throughout the specification. The system further comprises a database and at least one computing device in communication with at least one database and the user devices via a network. The database is configured to store client data, counselor data, assessment data including data related to first, second and master treatment plan, first score data, second score data and input data.

The computing device comprises an enhanced artificial intelligence (EAI). The computing device further comprises at least one memory configured to store a set of program modules and at least one processor. The processor is configured to execute one or more program modules. The modules comprise an assessment module, an upload module, a counseling module, an evaluation module, a treatment plan processing module, a treatment plan review module, and an approval module.

The assessment module is configured to enable the client to assess a treatment plan (TP). The assessed TP is a generated text file based on the specified goals, medical interventions, and follow-up of the client. The text file is converted into word using the enhanced artificial intelligence (EAI). The assessed treatment plan further comprises input data, including a self-assessment with a score provided by the client for a questionnaire comprising a plurality of categories to assess the recovery of the clients. The category comprises housing stability category, dosing consistency category, illicit substances category, counseling consistency category, physical and mental health category, legal issues impact category, medication management category, interpersonal relationships category, and renew gap category. The upload module is configured to upload the assessed treatment plan to the database.

The counseling module is configured to enable the counselor to review the assessed treatment plan and conduct an evaluation session with the client. The evaluation session is configured to review the self-assessment of the client and adjust the input and the score based on updated client data. The adjusted input includes qualitative notes having clinical verbiage, incorporates the enhanced artificial intelligence (EAI) technician comments and/alerts regarding concerns, and linkage metadata. The metadata is necessary for compliant storage and subsequent signature workflows. The metadata includes, but not limited to client identity and a National Provider Identifier (NPI) of the counselor.

The evaluation module is configured to receive the input data from the client for the questionnaire to assess the recovery of the clients. The evaluation module is further configured to determine a first score data based on the input data and notify the counselor if concerns are identified. Further, the evaluation module is configured to generate a second score data based on the evaluation session and the first score data.

The treatment plan processing module is configured to enable the administrator to review the assessed treatment plan and store the assessed treatment plan as a second treatment plan in a suitable format with respective counselor data in an encrypted system of the database. The treatment plan processing module is further configured to enable the administrator to verify the identity of the client using one or more authentication and verification processes for saving the assessed treatment plan. The authentication and verification processes includes verifying electronic health record (EHR) identity information, performing an authenticated portal login, executing a two-factor authentication, one-time password confirmation at signing, and conducting in-clinic verification when appropriate. The electronic health record (EHR) identity information comprises medical record number (MRN), identification (ID), and demographics. The treatment plan processing module is further configured to enable the administrator to verify the identity of the counselor using the National Provider Identifier (NPI).

The treatment plan processing module is further configured to enable the administrator to ensure proper format conversion of the treatment plan. The treatment plan processing module is further configured to enable the administrator to add one or more comments along with the second score data for counselor review. The comments are configured to support quality, compliance, and maintain workflow continuity of the assessed treatment plan.

The treatment plan review module is configured to enable the counselor to retrieve and assess the second treatment plan for clinical overlap. The treatment plan review module is further configured to integrate the second treatment plan into a master treatment plan for the client and enable the counselor to digitally sign the master treatment plan. The master treatment plan is a finalized and standardized clinic form, signed by the counselor. The treatment plan review module is further configured to flag the respective client for review and approval of the master treatment plan.

The approval module is configured to enable the administrator to open the flagged master TP for client review. The approval module is further configured to enable the client to approve the master treatment plan using an electronic signature. Further, the approval module is configured to store the finalized master TP in the database.

In one embodiment, a method for creating and managing treatment plan is disclosed. The method is executed in the system comprising one or more user devices, including at least one first user device associated with the client, at least one second user device associated with the clinician and the counselor, and at least one third user device associated with an administrator. The system further comprises the database and at least one computing device in communication with at least one database and the user devices via the network. The database is configured to store client data, counselor data, assessment data including data related to first, second and master treatment plan, first score data, second score data and input data.

The computing device comprises at least one memory configured to store the set of program modules and at least one processor. The processor is configured to execute one or more program modules. The modules comprise the assessment module, the upload module, the counseling module, the evaluation module, the treatment plan processing module, the treatment plan review module, and the approval module.

At one step, the assessment module, at the computing device, is configured to enable the client to assess the treatment plan (TP). At another step, the upload module, at the computing device, is configured to upload the assessed treatment plan to the database. The assessed treatment plan comprises the input data, including the self-assessment with the score provided by the client for the questionnaire comprising the plurality of categories to assess the recovery of the clients.

At yet another step, the counseling module, at the computing device, is configured to enable the counselor to review the assessed treatment plan and conduct the evaluation session with the client. The evaluation session is configured to review the self-assessment of the client and validate and adjust the input and the score based on updated client data.

At yet another step, the evaluation module, at the computing device, is configured to receive the input data from the client for the questionnaire to assess the recovery of the clients. At yet another step, the evaluation module, at the computing device, is configured to determine the first score data based on the input data and notify the counselor if concerns are identified. At yet another step, the evaluation module, at the computing device, is configured to generate the second score data based on the evaluation session and the first score data.

At yet another step, the treatment plan processing module, at the computing device, is configured to enable the administrator to review the assessed treatment plan and store the assessed treatment plan as the second treatment plan in the suitable format with respective counselor data in the encrypted system of the database.

At yet another step, the treatment plan processing module, at the computing device, is configured to enable the administrator to verify the identity of the client using one or more authentication and verification processes for saving the assessed treatment plan. At yet another step, the treatment plan processing module, at the computing device, is configured to verify the identity of the counselor using the National Provider Identifier (NPI) by the administrator. At yet another step, the treatment plan processing module, at the computing device, is configured to ensure proper format conversion of the treatment plan. At yet another step, the treatment plan processing module, at the computing device, is configured to enable the administrator to add comments along with the second score data for counselor review. The comments are configured to support quality, compliance, and maintain the workflow continuity of the assessed treatment plan.

At yet another step, the treatment plan review module, at the computing device, is configured to enable the counselor to retrieve and assess the second treatment plan for clinical overlap. At yet another step, the treatment plan review module, at the computing device, is configured to integrate the second treatment plan into the master treatment plan for the client and enable the counselor to digitally sign the master treatment plan. At yet another step, the treatment plan review module, at the computing device, is configured to flag the respective client for review and approval of the master treatment plan.

At yet another step, the approval module, at the computing device, is configured to enable the administrator to open the flagged master TP for client review. At yet another step, the approval module, at the computing device, is configured to enable the client to approve the master treatment plan using the electronic signature. At yet another step, the approval module, at the computing device, is configured to store the finalized master TP in the database.

Other objects, features and advantages of the present innovation will become apparent from the following detailed description. It should be understood, however, that the detailed description and the specific examples, while indicating specific embodiments of the innovation, are given by way of illustration only, since various changes and modifications within the spirit and scope of the innovation will become apparent to those skilled in the art from this detailed description.

### BRIEF DESCRIPTION OF THE DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates an environment of a system and method for creating and managing a treatment plan, according to an embodiment of the present invention.

FIG. 2 exemplarily illustrates a block diagram of the computing device in the system, according to an embodiment of the present invention.

FIG. 3 exemplarily illustrates a graph of R3 scoring based on the client data, according to an embodiment of the present invention.

FIG. 4 exemplarily illustrates a user interface displaying a graph of client distribution across two variable that includes illicit use and counseling consistency, according to an embodiment of the present invention.

FIG. 5 exemplarily illustrates a user interface displaying a radar chart and the R3 scores of a single client based on the client data, according to an embodiment of the present invention.

FIG. 6 exemplarily illustrates a flowchart of a method for creating an R3 EAI treatment plan (TP), according to an embodiment of the present invention.

FIG. 7 exemplarily illustrates an R3 index of 30 days cycle based on the counseling sessions attended by the client, according to an embodiment of the present invention.

### DETAILED DESCRIPTION OF EXAMPLE EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

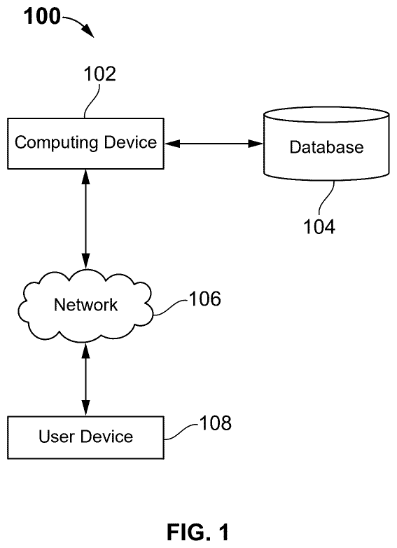

FIG. 1 exemplarily illustrates an environment 100 of a system and method for creating and managing a treatment plan, according to an embodiment of the present invention. In one embodiment, the system is utilized for creating and managing treatment plan for individuals related to substance use disorder (SUD). The system comprises at least one computing device 102, at least one database 104 in communication with the computing device 102 via a network 106. The system further comprises one or more user devices 108 in communication with the computing device 102 via the network 106. The user devices 108 include at least one first user device associated with a client, at least one second user device associated with a clinician and a counselor, and at least one third user device associated with an administrator. The clinician is also referred to as the counselor throughout the specification.

The user device 108 is configured to provide an interface to access the services provided by the computing device 102. The interface, for example, an application that allows the user device 108 to wirelessly connect and access the computing device 102 via the network 106. The user device 108 includes, but not limited to, a desktop computer, a laptop computer, a mobile phone, a personal digital assistant, and the like. The user includes a clinician, a client, and an administrator. In an example, the system is used in an android, an iOS, and a windows. The user device 108 is configured to provide one or more client data. The client is an individual related to substance use disorder (SUD). The client data is provided based on three categories. The three categories comprise a revive, a restore, a renew. Each category comprises one or more scoring guide inputs. Further, each scoring guide input is assigned a score value. The score value ranges from 1-10. Further, the system integrates the scoring methodologies with tailored AI workflows. The integration of AI-workflow with the structured scoring model helps to enhance operational efficiency of the system.

The revive category provides one or more information related to the housing stability, the dosing consistency, and the illicit substance. The housing stability is evaluated based on the information related to the housing stability status of the client. The dosing consistency is evaluated based on the information related to the number of doses missed in the past 30 days by the client. Further, the illicit substance is evaluated based on the information related to the amount of illicit substance usage by the client. Further, the R3 score is assigned by analyzing the information provided by the user via user device 108 for the revive category.

The restore category provides one or more information related to the counseling consistency, the physical and mental health, and the legal issues impact. The counseling consistency is evaluated based on the information related to the number of sessions missed in the past three months by the client. The physical and mental health is evaluated based on the information related to the physical and mental health of the client. Further, the legal issues impact is evaluated based on the information related to the legal issues involved by the client. Further, the R3 score is assigned by analyzing the information provided by the user via user device 108 for the restore category.

The renew category provides one or more information related to the renew gap, the interpersonal relationship, and the medication management. The renew gap is evaluated based on the information related to the number of questions the client answered with “No” in relation to client information. The interpersonal relationship is evaluated based on the information related to the relationship between the client and other people from family or friends. Further, the medication management is evaluated based on the information related to the duration taken continuous treatment and free from illicit drugs in the client. Further, the R3 score is assigned by analyzing the information provided by the user via user device 108 for the renew category.

The network 106 generally represents one or more interconnected networks, over which the user device 108, the computing device 102 could communicate with each other. The network 106 may include packet-based wide area networks (such as the Internet), local area networks (LAN), private networks, wireless networks, satellite networks, cellular networks, paging networks, and the like. A person skilled in the art will recognize that the network 106 may also be a combination of more than one type of network. For example, the network 106 may be a combination of a LAN and the Internet. In addition, the network 106 may be implemented as a wired network or a wireless network or a combination thereof.

In an example, the database 104 resides in the computing device 102. In another example, the database 104 resides separately from the computing device 102. Regardless of the location, the database 104 comprises a memory to store and organize data for use by the computing device 102. The database 104 comprises information related to the client with the substance use disorder (SUD) for use by the computing device 102. Further, the database 104 stores the R3 score assigned to the client data. The database 104 is further configured to store client data, counselor data, assessment data including data related to first, second and master treatment plan, first score data, second score data and input data.

In one embodiment, the computing device 102 is at least one of a server, a general-purpose computer, a special-purpose computer, a workstation, a desktop, a laptop, a tablet, a mobile phone, a mainframe, a supercomputer, and a server farm. Although the computing device 102 is illustrated as a single device, the functions performed by the computing device 102 could be performed using any suitable number of computing devices 102. The computing device comprises an enhanced artificial intelligence (EAI).

The computing device 102 is configured to enable the client to assess a treatment plan (TP). The assessed TP is a generated text file based on the specified goals, medical interventions, and follow-up of the client. The text file is converted into word document using the enhanced artificial intelligence (EAI). The assessed treatment plan comprises input data, including a self-assessment with a score provided by the client for a questionnaire comprising a plurality of categories to assess the recovery of the clients. Further, the computing device 102 is configured to analyze and assess a treatment plan (TP) for the client using an R3 enhanced artificial intelligence (EAI). The R3 Enhancing artificial intelligence (AI) helps to improve the capabilities to perform tasks, making decisions, and solving problems. Further, the R3 enhanced artificial intelligence (EAI) is designed to suit a highly customized artificial intelligence (AI) tool. Further, the EAI helps to support substance use disorder (SUD) treatment.

The EAI comprises an open-source large language models (LLMs), for example, Generative Pre-training Transformer (GPT) and is fine-tuned with domain-specific knowledge related to substance use disorder (SUD) treatment. Further, the EAI comprises one or more training data sources. The training data source includes guidelines and one or more policies from federal agencies, for example, the substance abuse and mental health services administration (SAMHSA) and drug enforcement administration (DEA). Further, the training data source also comprises industry-recognized evidence-based practices and state-level regulations, for example, LARA. The EAI further comprises one or more data relevant to SUD treatment and an extensive range of SUD-related documents. The SUD-related document includes, but not limited to, clinical guidelines, regulatory requirements, and best practice standards.

The computing device 102 enables the EAI to provide intelligent insights, recommendations, and workflow automation specific to the SUD recovery process. Further, the computing device 102 is configured to utilize algorithms tailored to SUD treatment requirements. The computing device 102 further utilizes the EAI for client assessment, treatment planning, and compliance monitoring of the information received by the user. The computing device 102 is configured to automate the documentation. The documentation includes, but not limited to, subjective, objective, assessment, and plan (SOAP) notes, treatment plan (TP) updates, and the use of a combination of structured data and unstructured data. For example, structured data is a client metric, and unstructured data is a clinician note. Additionally, the alignment of scoring and process automation improves scalability and consistency in SUD treatment.

The self-assessment comprises client inputs constitute that the first intake layer of assessment. A collaborative or clinical assessment layer follows during the counselor's evaluation session, where the counselor reviews the self-assessment with the client and may adjust the R3 score; this contributes to the second score data. Client “feedback” is therefore part of the self-assessment Questionnaire and the in-session discussion that informs the counselor-adjusted score and downstream plan. The client provides questionnaire responses mapped to the R3 categories and the subdomains, each scored on a scale of 1-10. The score provided by the client from the first score data is used to compute an initial R3 score and to trigger alerts if concerns are detected.

The computing device 102 is configured to upload the self-assessment into an existing electronic health records (EHR) system via a secure application programming interface (APIs), for example, Methasoft. The integration of the system with the existing electronic health records (EHR) systems ensures the real-time synchronization of the client data across multiple platforms. Further, the client is scheduled to meet the counselor either in a one-to-one session or by flagging the client in API. The counselor reviews the self-assessment and analyses with the client.

The computing device 102 is configured to enable the counselor to create a collaborative R3 score. The R3 score is assigned based on the client data provided via the user device 108. Further, the computing device 102 is configured to enable the counselor to adjust or modify the R3 score as needed. The modification could be based on new input data, progress in treatment, or changes in the client's situation. The computing device 102 is configured to enable the counselor to conduct an evaluation session reviewing the client's self-assessment with the client, once the self-assessment is uploaded. The counselor then creates a collaborative R3 score and adjusts the score based on updated situational data gathered live. The R3 EAI technician reviews score inputs and integrates them into the EAI system for TP generation.

The computing device 102 is configured to enable an R3 EAI technician to review the R3 score input and inform the counselor of the concerns of the client. Further, the R3 score and the comments are integrated into an R3 EAI system. The comments are configured to support quality, compliance, and maintain workflow continuity of the assessed treatment plan.

The computing device 102 is configured to create the treatment plan (TP) text file for the client. Further, the computing device 102 is configured to enable the R3 EAI technician to review the created TP text file. The computing device 102 is further configured to convert the TP text file into word document. Further, the computing device 102 is further configured to verify the client's identity. The computing device 102 is further configured to save the TP including a National Provider Identifier (NPI) number of the counselor in the file name. The computing device 102 is further configured to enable to store the TP in an encrypted system. Practically, the encrypted repository is part of the system architecture, for example, the encrypted database or storage module that is reachable by the computing device 102 over secure interfaces. The encrypted system might be co-located or separate, but is architecturally connected and operated as the system component. Further, the stored TP is reviewed by the counselor and the client. The computing device 102 is configured to incorporate a compliance focused process for secure data handling and treatment management.

The computing device 102 is further configured to enable the counselor to select the client's Treatment Plan (TP). The computing device 102 is further configured to enable the counselor to review the TP and conduct the evaluation session. The computing device 102 is further configured to receive client questionnaire inputs for plural categories. Further, the computing device 102 is configured to determine a first score data and notify the counselor of concerns. The computing device 102 is further configured to generate a second score data based on the evaluation session and the first score data. Further, the counselor is allowed to review TP for clinical verbiage and transfer TP to a master treatment plan form for the subject client. The master treatment plan is the finalized, counselor-signed plan integrated from the reviewed treatment plan, prepared in the standardized clinic form, and presented to the client for electronic approval. The master treatment plan is the authoritative plan used for ongoing care, stored in the encrypted system after client approval. Further, the counselor digitally signs the TP and flags the client to sign the TP as well. Flagging is an electronic status or action that makes the master TP available to the client for signature via the user device 108 or integrated through secure EHR APIs. The same concept of flagging is also used earlier for scheduling, for example, flagging the client in API. Operationally, this is a state change and notification in the system or EHR UI that prompts the client to open, review, and provide an electronic signature (e-sign).

The computing device 102 is configured to enable the client to open the flagged master treatment plan and review the master treatment plan. Further, the computing device 102 is configured to enable the client to approve the master treatment plan using an electronic signature pad. The computing device 102 is further configured to allow the client to save the master treatment plan for treating the client.

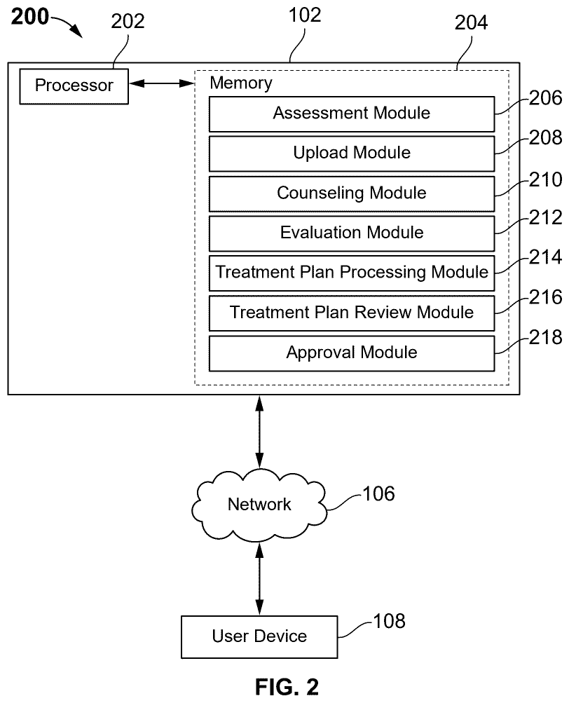

FIG. 2 exemplarily illustrates a block diagram 200 of the computing device 102 of the system, according to an embodiment of the present invention. The system comprises one or more user devices 108 including at least one first user device associated with the client, at least one second user device associated with the clinician and the counselor, and at least one third user device associated with an administrator. The system further comprises the database 104 and at least one computing device 102 in communication with at least one database 104 and the user devices 108 via the network 106. The database 104 is configured to store client data, counselor data, assessment data including data related to first, second and master treatment plan, first score data, second score data and input data.

The computing device comprises the enhanced artificial intelligence (EAI). The computing device 102 further comprises at least one processor 202 and at least one memory 204 in communication with the processor 202. The processor 202 is configured to execute one or more program modules. The program modules comprise an assessment module 206, an upload module 208, a counseling module 210, an evaluation module 212, a treatment plan processing module 214, a treatment plan review module 216, and an approval module 218.

The assessment module 206 is configured to enable the client to assess the treatment plan (TP). The assessed TP is the generated text file based on the specified goals, medical interventions, and follow-up of the client. The text file is converted into word document using the enhanced artificial intelligence (EAI). The assessed treatment plan further comprises the input data, including the self-assessment with the score provided by the client for the questionnaire comprising the plurality of categories to assess the recovery of the clients. The category comprises housing stability category, dosing consistency category, illicit substances category, counseling consistency category, physical and mental health category, legal issues impact category, medication management category, interpersonal relationships category, and renew gap category. The upload module 208 is configured to upload the assessed treatment plan to the database 104.

The self-assessment related to the client provided by the client via the user device 108, across the R3 categories, each composed of scoring guide inputs rated 1-10. Further, the self-assessment is uploaded to the existing electronic health records (EHR) systems via the secure application programming interface (APIs), for example, Methasoft.

The counseling module 210 is configured to enable the counselor to review the assessed treatment plan and conduct the evaluation session with the client. The evaluation session is configured to review the self-assessment of the client and adjust the input and the score based on updated client data. The updated client data could be based on new input data, progress in treatment, or changes in the client's situation. The adjusted input includes qualitative notes having clinical verbiage, incorporates the enhanced artificial intelligence (EAI) technician comments and/alerts regarding concerns, and linkage metadata. The metadata is necessary for compliant storage and subsequent signature workflows. The metadata includes client identity and a National Provider Identifier (NPI) of the counselor.

The evaluation module 212 is configured to receive the input data from the client for the questionnaire to assess the recovery of the clients. The evaluation module 212 is further configured to determine the first score data based on the input data and notify the counselor if concerns are identified. Further, the evaluation module 212 is configured to generate the second score data based on the evaluation session and the first score data.

The treatment plan processing module 214 is configured to enable the administrator to review the assessed treatment plan and store the assessed treatment plan as a second treatment plan in a suitable format with respective counselor data in the encrypted system of the database 104. The treatment plan processing module 214 is further configured to enable the administrator to verify the identity of the client using one or more authentication and verification processes for saving the assessed treatment plan. The authentication and verification processes include verifying electronic health record (EHR) identity information, performing an authenticated portal login, executing a two-factor authentication, one-time password confirmation at signing, and conducting in-clinic verification when appropriate. The electronic health record (EHR) identity information comprises medical record number (MRN), identification (ID), and demographics.

The treatment plan processing module 214 is further configured to enable the administrator to verify the identity of the counselor using the National Provider Identifier (NPI). The treatment plan processing module 214 is further configured to enable the administrator to ensure proper format conversion of the treatment plan. The treatment plan processing module 214 is further configured to enable the administrator to add one or more comments along with the second score data for counselor review. The comments are configured to support quality, compliance, and maintain workflow continuity of the assessed treatment plan. For example, data quality notes including incomplete fields or conflicting inputs, regulatory or compliance reminders, requests for counselor clarification on clinical verbiage, risk or concern annotations surfaced to the counselor, and scheduling or follow-up instructions that inform the EAI technician and counselor prior to finalization. The comments are retained with the TP or score record in the encrypted system.

The treatment plan processing module 214 is configured to create the treatment plan text file based on the R3 score. The treatment plan processing module 214 is configured to ensure to review the TP text file and convert the TP text file into the word document. Further, the treatment plan processing module 214 is configured to save the TP including the National Provider Identifier (NPI) number of the counselor in the file name.

The treatment plan review module 216 is configured to enable the counselor to retrieve and assess the second treatment plan for clinical overlap. The treatment plan review module 216 is further configured to integrate the second treatment plan into the master treatment plan for the client and enable the counselor to digitally sign the master treatment plan. The master treatment plan is a finalized and standardized clinic form, signed by the counselor. The treatment plan review module 216 is further configured to flag the respective client for review and approval of the master treatment plan.

The treatment plan review module 216 is configured to enable the counselor to review the self-assessment and the R3 score and to create the collaborative R3 score. Further, the treatment plan review module 216 is configured to enable the counselor to select and review the TP word document for clinical verbiage. Further, the counselor transfers the TP document into the master TP form for the subject client. Further, the counselor digitally signs the master TP and forwards the master TP to the client.

The approval module 218 is configured to enable the administrator to open the flagged master TP for client review. The approval module 218 is further configured to enable the client to approve the master treatment plan using the electronic signature. The approval module 218 is further configured to enable the administrator to open the flagged master TP for client review. Further, the approval module 218 is configured to store the finalized master TP in the database 104. The stored TP embeds the counselor's NPI in the filename and resides in the encrypted repository.

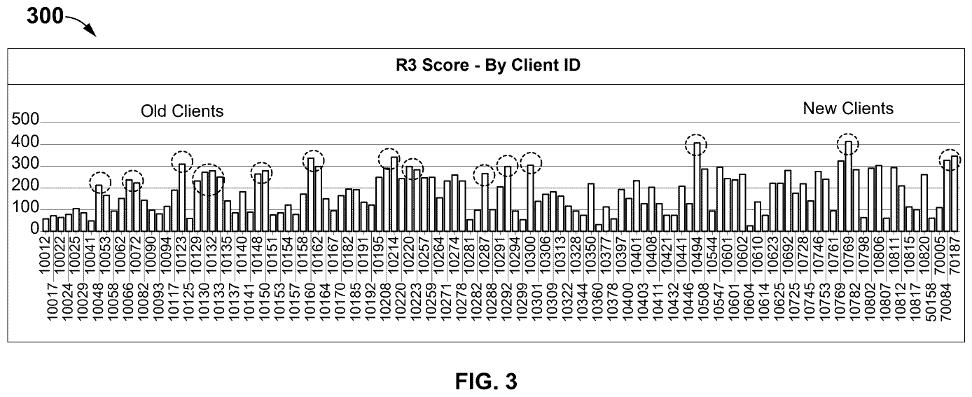

FIG. 3 exemplarily illustrates a graph 300 of R3 scoring based on the client data, according to an embodiment of the present invention. The graph is provided by the sum of R3 scoring provided to each client ID. The R3 scores help the clinicians to focus on recovery, progress, and results in addition to the administrative compliance of the client. Further, the R3 scoring provides a quantitative understanding for both the client and the clinician to understand the TP. Further, the quantitative understanding helps to discuss and adjust the client's treatment plan.

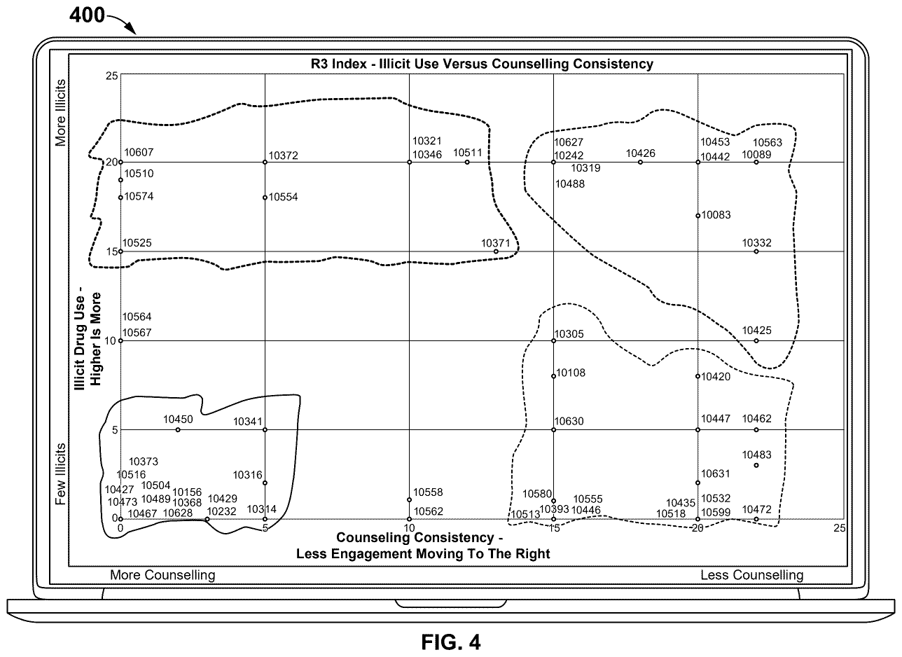

FIG. 4 exemplarily illustrates a user interface 400 displaying a graph of client distribution across two variable that includes an illicit use and a counseling consistency, according to an embodiment of the present invention. The graph plotted based on two variable enables the organization to identify client segments with similar needs. Further, the data analysis creates four distinct quadrants. The four distinct quadrants comprise a first quadrant, a second quadrant, a third quadrant, and a fourth quadrant. Each quadrant representing a unique combination of behavior related to the illicit use and the counseling consistency. The first quadrant represents clients with high illicit use and high counseling consistency. Clients in the first quadrant are actively using illicit substances and attend counseling regularly. The second quadrant represents clients with high illicit use and low counseling consistency. Clients in the second quadrant are actively using illicit substances and have low engagement in counseling. The third quadrant represents clients with low illicit use and high counseling consistency. Clients in the third quadrant are actively attending the counseling sessions and exhibit low illicit use. The fourth quadrant represents clients with low illicit use and low counseling consistency. Clients in the fourth quadrant are inconsistent in attending counseling and have low illicit use.

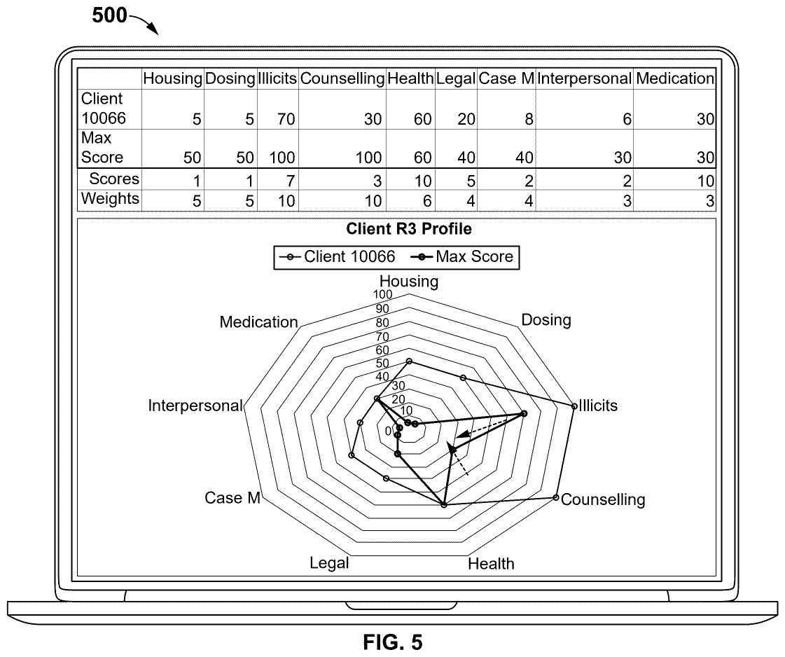

FIG. 5 exemplarily illustrates a user interface 500 displaying a radar chart and the R3 scores of a single client based on the client data, according to an embodiment of the present invention. Radar charts enable precise communication of client challenges using graphical tools. The chart visually represents various aspects of the client's behavior. The smaller the shape of the chart, the better the behavior. Further, the R3 scoring is given based on client data related to the housing stability, the dosing consistency, the illicit substance, counseling consistency, the physical and mental health, the legal issues impact, the renew gap referred as case M, the interpersonal relationship, and the medication management.

In the given case, the R3 score for the housing stability of the client 10066 is 5 out of a maximum score is 50, where the score value is 1 and the weight is 5. The R3 score for the dosing consistency of the client 10066 is 5 out of the maximum score is 50, where the score value is 1 and the weight is 5. The R3 score for the illicit substance of the client 10066 is 70 out of the maximum score is 100, where the score value is 7 and the weight is 10.

In the given case, the R3 score for the counseling consistency of the client 10066 is 30 out of the maximum score is 100, where the score value is 3 and the weight is 10. The R3 score for the physical and mental health of the client 10066 is 60 out of the maximum score is 60, where the score value is 10 and the weight is 6. The R3 score for the legal issues impact of the client 10066 is 20 out of the maximum score is 40, where the score value is 5 and the weight is 4.

In the given case, the R3 score for the renew gap or case M of the client 10066 is 8 out of the maximum score is 40, where the score value is 2 and the weight is 4. The R3 score for the interpersonal relationship of the client 10066 is 6 out of the maximum score is 30, where the score value is 2 and the weight is 3. The R3 score for the medication management of the client 10066 is 30 out of the maximum score is 30, where the score value is 10 and the weight is 3. The client 10066 exhibits health issues and challenges with illicit drug use. However, the client-counselor engagement is showing improvement.

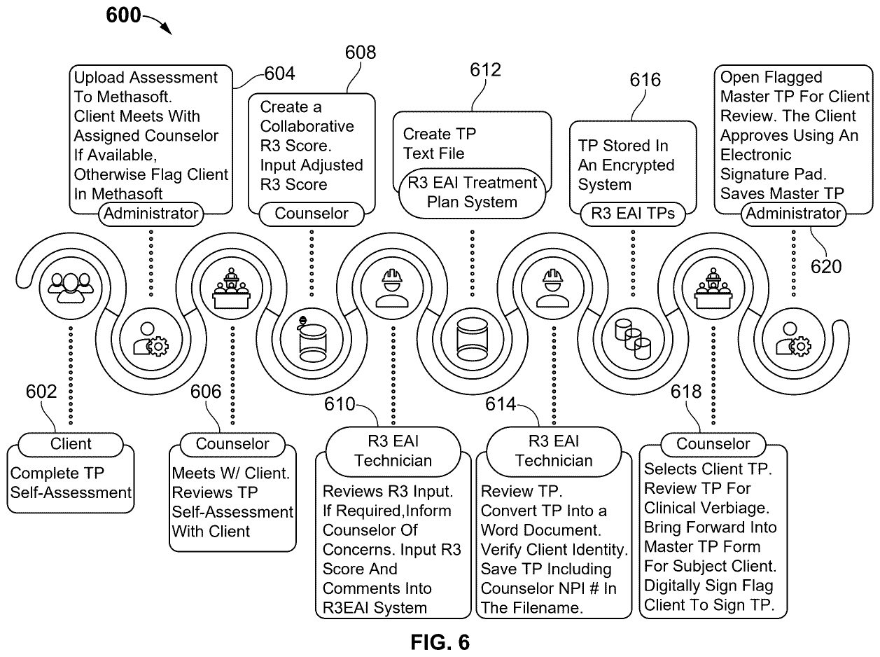

FIG. 6 exemplarily illustrates a flowchart of a method 600 to create an R3 EAI treatment plan (TP), according to an embodiment of the present invention. The method 600 is incorporated in the system comprising the computing device 102, the database 104 in communication with the computing device 102. The system further comprises the user device 108 including at least one first user device associated with the client, at least one second user device associated with the clinician and the counselor, and at least one third user device associated with the administrator. The user device 108 and the database 104 are in communication with the computing device 102 via the network 106.

At step 602, the computing device 102 is configured to assess the treatment plan (TP) and complete the treatment plan (TP). The assessed treatment plan comprises input data, including a self-assessment with a score provided by the client for a questionnaire comprising a plurality of categories to assess the recovery of the clients. At step 604, the computing device 102 is configured to enable the administrator to upload the assessed treatment plan, including self-assessment with the score into the existing electronic health records (EHR) systems via the secure application programming interface (APIs), for example, Methasoft. Further, the computing device 102 is configured to enable the administrator to schedule a meeting for the counselor and client either in the one-to-one session or by flagging the client in API.

At step 606, the computing device 102 is configured to enable the counselor to review the assessed treatment plan and conduct the evaluation session with the client. The evaluation session is configured to review the treatment plan of the client and adjust the input and the score based on updated client data.

At step 608, the computing device 102 is configured to enable the counselor to create a collaborative R3 score. Further, the computing device 102 allows the counselor to adjust the R3 score based on the updated client's information. The updated information is based on the current situation of the client.

At step 610, the computing device 102 is configured to enable the R3 EAI technician to review the R3 input and inform the counselor of the concerns of the client. Further, the R3 EAI technician integrates the R3 score and the comments into the R3 EAI system.

At step 612, the computing device 102 is configured to enable an R3 EAI treatment plan system to create the treatment plan (TP) text file for the client.

At step 614, the computing device 102 is configured to enable the R3 EAI technician to review the created TP text file. Further, the computing device 102 is configured to enable the R3 EAI technician convert the TP text file into the word document. The computing device 102 is further configured to verify the client identity and save the TP including the National Provider Identifier (NPI) number of the counselor in the file name.

At step 616, the computing device 102 is further configured to store the TP in an encrypted system. The stored TP embeds the counselor's NPI in the filename and resides in an encrypted repository.

At step 618, the computing device 102 is configured to enable the counselor to select the client TP and review the client TP for clinical verbiage. The client TP is also referred as the second treatment plan. The client TP is forwarded to the master TP form for the subject client. Further, the computing device 102 allows the counselor to digitally sign the master TP and flag the client to sign the master TP.

At step 620, the computing device 102 is configured to enable the administrator to open the flagged master treatment plan and allow the client to review the master treatment plan. Further, the computing device 102 is configured to enable the client to approve the master treatment plan using the electronic signature. The computing device 102 further allows the administrator to save the master treatment plan to the database 104 for treating the client.

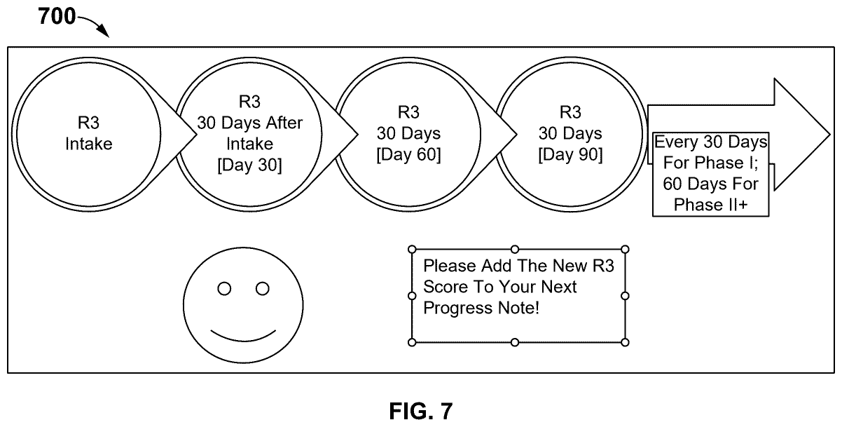

FIG. 7 exemplarily illustrates a R3 index of 30 days cycle 700 based on the counseling sessions attended by the client, according to an embodiment of the present invention. The cycles are observed after every 30 days during the counselling or the treatment process. The R3 score is noted for every 30 days sequence of the treatment that includes 30 days, 60 days, and 90 days. Usually, the R3 score is noted for the clients under phase I is 30 days and for the clients under phase II+ is 60 days. Further, the new R3 score is noted for every progress that is observed in the client.

Advantageously, the system is integrated with the existing electronic health records (EHR) system. The integration ensures the real-time synchronization of the client data across multiple platforms. Further, the integration supports streamlined workflows and ensures the data used for R3/R4 Index assessments is always accurate. Further, the system is compatible with both iOS and android devices, which enables the user to access the system across different devices. The system automates key documentation tasks and provides AI-driven suggestions for SOAP notes and treatment plans. The automation function reduces the administrative burden on clinicians and improves workflow efficiency.

The system incorporates the data and guidelines from federal and state regulatory bodies, for example, DEA, LARA, and SAMHSA into the AI logic. The integration ensures that the recommendations generated are not only evidence-based but also fully compliant with regulatory requirements. The system integrates the technical and functional features into a scalable, compliance-driven workflow. Further, the system ensures secure, automated, and effective treatment management through streamlined workflows.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device, or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A system for creating and managing treatment plan, comprising:
one or more user devices including at least one first user device associated with a client, at least one second user device associated with a clinician and a counselor, and at least one third user device associated with an administrator;
a database configured to store client data, counselor data, assessment data including data related to first, second and master treatment plan, first score data, second score data and input data, and
at least one computing device in communication with at least one database and the user devices via a network, wherein the computing device comprises an enhanced artificial intelligence (EAI), wherein the computing device further comprises at least one memory configured to store a set of program modules and at least one processor configured to execute one or more program modules, wherein the modules comprise:
an assessment module configured to enable the client to assess a treatment plan (TP), wherein the assessed TP is a generated text file based on the specified goals, medical interventions, and follow-up of the client, wherein the text file is converted into word document using the enhanced artificial intelligence (EAI), wherein the assessed treatment plan comprises input data including a self-assessment with a score provided by the client for a questionnaire comprising a plurality of categories to assess the recovery of the clients;
an upload module configured to upload the assessed treatment plan to the database;
a counseling module configured to enable the counselor to review the assessed treatment plan and conduct an evaluation session with the client, wherein the evaluation session is configured to review the self-assessment of the client and adjust the input and the score based on updated client data;
an evaluation module configured to:
receive the input data from the client for the questionnaire to assess the recovery of the clients;
determine a first score data based on the input data and notify the counselor if concerns are identified, and
generate a second score data based on the evaluation session and the first score data,
a treatment plan processing module configured to enable the administrator to review the assessed treatment plan and store the assessed treatment plan as a second treatment plan in a suitable format with respective counselor data in an encrypted system of the database, and
a treatment plan review module configured to:
enable the counselor to retrieve and assess the second treatment plan for clinical overlap;
integrate the second treatment plan into a master treatment plan for the client and enable the counselor to digitally sign the master treatment plan, wherein the master treatment plan is a finalized and standardized clinic form, signed by the counselor and
flag the respective client for review and approval of the master treatment plan, and
an approval module is configured to enable the client to approve the master treatment plan using an electronic signature.

2. The system of claim 1, wherein the treatment plan processing module is configured to enable the administrator to:
verify the identity of the client using one or more authentication and verification processes for saving the assessed treatment plan;
verify the identity of the counselor using a National Provider Identifier (NPI);
ensure proper format conversion of the treatment plan, and
add one or more comments along with the second score data for counselor review, wherein the comments are configured to support quality, compliance, and maintain workflow continuity of the assessed treatment plan.

3. The system of claim 1, wherein the adjusted input includes qualitative notes having clinical verbiage, incorporates the enhanced artificial intelligence (EAI) technician comments and/alerts regarding concerns, and linkage metadata, wherein the metadata includes client identity and counselor NPI necessary for compliant storage and subsequent signature workflows.

4. The system of claim 1, wherein the approval module is configured to enable the administrator to open the flagged master TP for client review.

5. The system of claim 1, wherein the approval module is configured to store the finalized master TP in the database.

6. The system of claim 1, wherein the authentication and verification processes includes verifying electronic health record (EHR) identity information, performing an authenticated portal login, executing a two-factor authentication, one-time password confirmation at signing, and conducting in-clinic verification when appropriate, wherein the electronic health record (EHR) identity information comprises medical record number (MRN), identification (ID), and demographics.

7. The system of claim 1, wherein the category comprises housing stability category, dosing consistency category, illicit substances category, counseling consistency category, physical and mental health category, legal issues impact category, medication management category, interpersonal relationships category, and renew gap category.

8. A method for creating and managing treatment plan, comprising the steps:
providing one or more user devices, a database and at least one computing device in communication with at least one database and the user devices via a network, wherein the user devices include at least one first user device associated with a client, at least one second user device associated with a clinician and a counselor, and at least one third user device associated with an administrator, wherein the database is configured to store client data, counselor data, assessment data including data related to first, second and master treatment plan, first score data, second score data and input data, wherein the computing device comprises an enhanced artificial intelligence (EAI), wherein the computing device further comprises at least one memory configured to store a set of program modules and at least one processor configured to execute one or more program modules;
enabling, by the computing device via an assessment module, the client to assess a treatment plan (TP), wherein the assessed treatment plan comprises input data including a self-assessment with a score provided by the client for a questionnaire comprising a plurality of categories to assess the recovery of the clients;
uploading, by the computing device via an upload module, the assessed treatment plan to the database;
enabling, by the computing device via a counseling module, the counselor to review the assessed treatment plan and conduct an evaluation session with the client, wherein the evaluation session is configured to review the self-assessment of the client and validate and adjust the input and the score based on updated client data;
receiving, by the computing device via an evaluation module, the input data from the client for the questionnaire to assess the recovery of the clients;
determining, by the computing device via the evaluation module, a first score data based on the input data and notifying the counselor if concerns are identified;
generating, by the computing device via the evaluation module, a second score data based on the evaluation session and the first score data;
enabling, by the computing device via a treatment plan processing module, the administrator to review the assessed treatment plan and store the assessed treatment plan as a second treatment plan in a suitable format with respective counselor data in an encrypted system of the database;
enabling, by the computing device via a treatment plan review module, the counselor to retrieve and assess the second treatment plan for clinical overlap;
integrating, by the computing device via the treatment plan review module, the second treatment plan into a master treatment plan for the client, and enable the counselor to digitally sign the master treatment plan;
flagging, by the computing device via the treatment plan review module, the respective client for review and approval of the master treatment plan, and
enabling, by the computing device via an approval module, the client to approve the master treatment plan using an electronic signature.

9. The method of claim 8, further comprising a step of: enabling, by the computing device via the approval module, the administrator to open the flagged master TP for client review.

10. The method of claim 8, further comprising a step of: storing, by the computing device via the approval module, the finalized master TP in the database.

11. The method of claim 8, further comprising a step of: enabling, by the computing device via the treatment plan processing module, the administrator to verify the identity of the client using one or more authentication and verification processes for saving the assessed treatment plan.

12. The method of claim 8, further comprising a step of: verifying, by the computing device via the treatment plan processing module, the identity of the counselor using a National Provider Identifier (NPI) by the administrator.

13. The method of claim 8, further comprising a step of: ensuring, by the computing device via the treatment plan processing module, proper format conversion of the treatment plan.

14. The method of claim 8, further comprising a step of: enabling, by the computing device via the treatment plan processing module, the administrator to add comments along with the second score data for counselor review, wherein the comments are configured to support quality, compliance, and maintain workflow continuity of the assessed treatment plan.

15. The method of claim 8, wherein the category comprises housing stability category, dosing consistency category, illicit substances category, counseling consistency category, physical and mental health category, legal issues impact category, medication management category, interpersonal relationships category, and renew gap category.
